(19) United States
(12) Patent Application Publication
(10) Pub. No.: US 2024/0261217 A1
(43) Pub. Date: Aug. 8, 2024

(54) NUTRACEUTICAL GUMMY COMPOSITIONS AND METHOD OF PREVENTING ALCOHOL OVERCONSUMPTION SYMPTOMS

(71) Applicant: LiQure, Inc., Chicago, IL (US)

(72) Inventors: Daniel Arthur Adix, Chicago, IL (US); Chad Brey, Bakersfield, CA (US)

(73) Assignee: LiQure, Inc., Chicago, IL (US)

(21) Appl. No.: 18/435,964

(22) Filed: Feb. 7, 2024

Related U.S. Application Data
(60) Provisional application No. 63/483,852, filed on Feb. 8, 2023.

(51) Int. Cl. A61K 9/00; A61K 31/198; A61K 31/4415; A61K 31/714; A61K 33/00; A61K 36/33; A61K 36/87; A61K 36/906

(52) U.S. Cl. CPC A61K 9/0056; A61K 31/198; A61K 31/4415; A61K 31/714; A61K 33/00; A61K 36/33; A61K 36/87; A61K 36/906

(57) ABSTRACT

A nutraceutical gummy composition is disclosed for counteracting effects of alcohol-containing beverages and solid food products. The composition includes electrolytes, vitamin B complexes, and liver detoxification compounds. Specific components include ampelopsis grossedentata extract (DHM), prickly pear cactus extract, milk thistle extract, ginger root extract, and L-alanyl-L-glutamine. The composition also includes a gummy base and a coating, with the base comprising citrus pectin and water, and the coating comprising safflower oil and carnauba wax. The L-alanyl-L-glutamine component is particularly effective in reducing a glutamine rebound in a user. The composition is designed to be consumed by a subject to treat effects of alcohol consumption.

### CROSS-REFERENCE TO RELATED APPLICATION(S)

This application claims the benefit of priority under 35 U.S.C. §§ 120 and 119(e) of U.S. provisional application No. 63/483,852, filed Feb. 8, 2023, entitled “NUTRACEUTICAL GUMMY COMPOSITIONS AND METHOD OF PREVENTING ALCOHOLOVERCONSUMPTION SYMPTOMS.”

The contents of each of the above referenced application(s) is hereby incorporated by reference in its entirety.

### TECHNICAL FIELD

The present subject matter is directed, in general to a gummy nutraceutical and, more particularly, is directed to nutraceutical gummy compositions for use in a method for prevention or alleviation of symptoms related to the over-consumption of alcohol.

### BACKGROUND

Dehydration can result from overconsumption of alcoholic beverages by “of age” adults, with some of them even experiencing a hangover resulting from over-intoxication. These conditions not only negatively impact the health of one's liver and other organs of a person's body but also one's ability to function efficiently and productively the next day.

Dihydromyricetin (DHM) is an ingredient found in certain after-alcoholic-consumption products. DHM, an extract from Japanese grape seed trees, has been used as an anti-hangover herb and hangover treatment in traditional medicine in Korea and China. Moreover, recent studies have shown that DHM can reduce blood alcohol levels and protect the liver from alcohol-induced damage. Studies and anecdotal reports have also shown, when taking DHM after drinking alcohol, that users experience fewer headaches, less nausea, less vomiting and less anxiety. Scientists at the University of California, Los Angeles and the USC School of Pharmacy conducted a study in which DHM was administered to intoxicated mice. Those results demonstrated that DHM possesses liver-protection properties as well as being able to provide “anti-hangover” benefits. See Shen Y; Lindemeyer A K; Gonzalez C; Shao X M; Spigelman I; Olsen R W; and Liang J: “Dihydromyricetin as novel anti-alcohol intoxication medication,” J Neurosci 2012, 32(1):390-401, the entire contents of which are hereby incorporated by reference.

Two enzymes—alcohol dehydrogenase (ADH) and acetaldehyde dehydrogenase (ALDH)—facilitate bodily breakdown of alcohol. Studies have shown that DHM enhances the activity of these enzymes, suggesting that DHM may help a person's body metabolize alcohol faster. See Chen S H; Zhong G S; Li A L; Li S H; and Wu L K: “Influence of Hovenia dulcis on alcohol concentration in blood and activity of alcohol dehydrogenase (ADH) of animals after drinking,” Zhongguo Zhong Yao Za Zhi 2006, 31 (13): 1094-1096, the entire contents of which are hereby incorporated by reference. Thus, the sooner blood alcohol levels reach zero, the sooner hangover symptoms will disappear. A recent study has shown, compared with people who did not take DHM, that those who took DHM had fewer headaches, less dizziness, less nausea, and less weakness during a hangover. See Kim H; Kim Y J; Jeong H Y; Kim J Y; Choi E K; Chae S W; and Kwon O: “A standardized extract of the fruit of Hovenia dulcis alleviated alcohol-induced hangover in healthy subjects with heterozygous ALDH2: A randomized, controlled, crossover trial,” J Ethnopharmacol 2017, 209: 167-174, the entire contents of which are hereby incorporated by reference.

Besides the anti-alcoholic effects of DHM, studies have demonstrated that DHM can also prevent (and help repair) alcohol-induced liver damage. See Xiang J; Zhu W; Li Z; and Ling S: “Effect of juice and fermented vinegar from Hovenia dulcis peduncles on chronically alcohol-induced liver damage in mice,” Food Funct 2012, 3(6): 628-634; and Fang H L; Lin H Y; Chan M C; Lin W L; and Lin W C: “Treatment of chronic liver injuries in mice by oral administration of ethanolic extract of the fruit of Hovenia dulcis, “Am J Chin Med 2007, 35(4): 693-703, the entire contents of all of which are hereby incorporated by reference. Accordingly, it is believed these findings support the effectiveness of DHM as a treatment to relieve, alleviate, or ameliorate the symptoms of a hangover, and to prevent (or reduce) liver damage induced by ethanol—by changing lipid metabolism, enhancing ethanol metabolism, and inhibiting inflammatory reaction-thereby promoting liver health. It is also believed that DHM may also help people having early warning signs of liver injury. As such, it is believed that DHM can be used to help restore and prolong liver function, and delay (or prevent or reduce the likelihood of) the occurrence of liver disease.

Currently available DHM-containing compositions are unpleasant or are inconvenient to carry and consume, for example, during social events.

Nutraceutical products are frequently sold as gummies such as gummy bears, since they are a convenient and pleasant means of composition ingestion. The term “gummy bear” originated in Germany. The original base ingredient used to produce gummy bears was gum arabic. Current gummy bears are now made from a mixture of sugar, glucose syrup, starch, flavoring, food coloring, citric acid, and gelatin. Recipes vary and include ingredients suitable for vegetarians or those following religious dietary laws.

In spite of the benefits of DHM and of gummies as a delivery means, there are no currently available DHM products in a “gummy”-based carrier. It has been considered common knowledge that DHM cannot be provided in a gummy base at a concentration suitable to have any effect without ingesting vast quantities of a gummy product.

The present disclosure is directed to overcoming one or more of these above-referenced challenges.

### SUMMARY OF THE DISCLOSURE

The present subject matter is directed to a gummy-based product to be taken orally by a person to counteract intoxicating effects of alcohol-containing beverages and solid food products. The term “alcohol” as used throughout this patent specification shall refer to a known composition, commonly known as ethanol, which is fit for human consumption.

The gummy-based products of the present disclosure are to be taken orally by a person to counteract intoxicating effects of alcohol-containing beverages and other food products ingested by a person by relieving, alleviating, or ameliorating symptoms of a hangover and preventing or reducing liver damage otherwise induced by ethanol, since the gummy-based products include an effective amount of dihydromyricetin for doing so.

In some cases, a nutraceutical gummy composition for counteracting effects of alcohol-containing beverages and solid food products may include: electrolytes; vitamin B complexes; liver detoxification compounds; wherein the composition includes ampelopsis grossedentata extract (DHM), prickly pear cactus extract, milk thistle extract, ginger root extract, and L-alanyl-L-glutamine.

In some cases, a nutraceutical composition for counteracting effects of alcohol-containing beverages and solid food products may include: electrolytes; vitamin B complexes; liver detoxification compounds; wherein the composition includes ampelopsis grossedentata extract (DHM), prickly pear cactus extract, milk thistle extract, ginger root extract, and L-alanyl-L-glutamine, and the composition includes a gummy base and a coating, wherein the gummy base includes citrus pectin and water, and the coating includes an oil and a wax.

In some cases, a method of treating effects of alcohol-containing beverages and solid food products in a subject may include: causing a subject to consume a nutraceutical gummy composition. The nutraceutical gummy composition includes: electrolytes; vitamin B complexes; liver detoxification compounds; wherein the composition includes ampelopsis grossedentata extract, prickly pear cactus extract, milk thistle extract, ginger root extract, and L-alanyl-L-glutamine, and an amount of the ampelopsis grossedentata extract (minimum 98% dihydromyricetin) is an effective amount for counteracting intoxicating effects.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### BRIEF DESCRIPTION OF THE DRAWINGS

The accompanying drawings, which are incorporated in and constitute a part of this specification, illustrate various exemplary aspects and together with the description, serve to explain the principles of the disclosed technology.

FIG. 1 is a diagram of gummy-based products according to an embodiment of the present disclosure; and

FIG. 2 is a flow diagram of ingredient interactions thereof.

FIG. 3 is a diagram of gummy-based products according to an embodiment of the present disclosure.

### DETAILED DESCRIPTION

The following detailed description is of the best currently contemplated modes of carrying out exemplary embodiments of the disclosure. The description is not to be taken in a limiting sense but is made merely for the purpose of illustrating the general principles of the disclosure, since the scope of the disclosure is best defined by the appended claims.

In some embodiments, the composition may comprise about 31.032 weight-percent (wt.- %) apple puree, about 17.068 wt.- % allulose syrup, about 12.102 wt.- % raw cane sugar, about 10.473 wt.- % distilled or deionized water, about 7.293 wt.- % erythritol, about 6.206 wt.- % pectin, about 5.974 wt. % Active Pharmaceutical Ingredient (API) masterbatch, about 2.638 wt.- % glucomannan, about 2.638 wt.- % stevia, about 2.638 wt.- % bitter blocker, about 1.552 wt.- % citric acid, about 0.233 wt.- % trisodium citrate, and about 0.155 wt.- % riboflavin.

In some embodiments, the composition may comprise about 1 gram (g) dietary fiber, about 3 g total sugars, about 3 g sugar alcohols, about 10 mg vitamin B6 (as pyridoxine HCL), about 500 mcg vitamin B12 (as cyanocobalamin), about 50 mg vitamin B1 (as thiamine HCL), about 20 mg calcium (as carbonate, citrate), about 10 mg magnesium (as bisglycinate, oxide), about 30 mg potassium (as potassium phosphate), about 10 mg phosphorous (as potassium phosphate), about 8 mg sodium (as sodium chloride), about 19 mg chloride (as sodium chloride), about 100 mg prickly pear cactus (Opuntia ficus-indica) leaf, about 80 mg milk thistle (Silybum marianum) (minimum 80% silymarin), about 300 mg Ampelopsis grossedentata extract (minimum 98% dihydromyricetin), about 75 mg ginger (Zingiber officinale) (5% minimum gingerols and shogaol), and about 50 mg L-alanyl-L-glutamine.

### Dihydromyricetin (DHM)

In some cases, the composition of the present disclosure may include dihydromyricetin (DHM). The dihydromyricetin (DHM) in the composition may be derived from the ampelopsis grossedentata plant. Dihydromyricetin has been found as a novel anti-alcohol intoxication agent. See, for instance, Shen Y, Lindemeyer A K, Gonzalez C, Shao X M, Spigelman I, Olsen R W, Liang J. Dihydromyricetin as a novel anti-alcohol intoxication medication. J Neurosci. 2012 Jan. 4; 32(1):390-401. doi: 10.1523/JNEUROSCI.4639-11.2012. PMID: 22219299; PMCID: PMC3292407 (available at https://www.ncbi.nlm.nih.gov/pmc/articles/PMC3292407/). Dihydromyricetin in ranges of 100-650 mg of have been found to mitigate symptoms of over-intoxication from alcohol. See, for instance, Shen above. The compositions of the present disclosure may use the range of 200-450 mg to maximize the ratio of effectiveness on alcohol-related symptoms, while maintaining the flavor & palatability of the gummy medium.

DHM, or dihydromyricetin, is a pivotal component of the nutraceutical composition that works in concert with other ingredients to combat the effects of a hangover. DHM is known for its ability to reduce blood alcohol levels and protect liver cells from alcohol-induced damage. It does this by enhancing the activity of enzymes such as alcohol dehydrogenase (ADH) and acetaldehyde dehydrogenase (ALDH), which are involved in the metabolism of alcohol, thereby accelerating the breakdown and elimination of alcohol from the body.

When combined with other liver-supportive ingredients like milk thistle and prickly pear cactus extract, DHM contributes to a comprehensive liver detoxification process. Milk thistle contains silymarin, which is known for its liver-protective and regenerative properties, while prickly pear cactus is rich in antioxidants that help reduce oxidative stress and inflammation in the liver.

Additionally, the inclusion of ginger root extract in the composition provides anti-inflammatory and anti-nausea benefits, which can alleviate gastrointestinal symptoms commonly associated with hangovers. The amino acid L-alanyl-L-glutamine helps to regulate the body's glutamine levels, which can be disrupted after alcohol consumption, promoting better sleep and recovery.

Together, these ingredients work synergistically to reduce the toxic effects of alcohol, support liver health, and alleviate the various symptoms of a hangover, providing a more holistic approach to hangover management.

### Milk Thistle and Prickly Pear Cactus

In some cases, the composition of the present disclosure may include milk thistle (Silybum marianum) seed extract and prickly pear cactus (Opuntia ficus-indica) extract. The milk thistle (Silybum marianum) seed extract and prickly pear cactus (Opuntia ficus-Indica) extract may work synergistically together with their individual antioxidant & anti-inflammatory properties, providing comprehensive liver support and hangover relief. See, for instance, Wang F, Li Y, Zhang Y J, Zhou Y, Li S, Li H B. Natural Products for the Prevention and Treatment of Hangover and Alcohol Use Disorder. Molecules. 2016 Jan. 7; 21(1):64. doi: 10.3390/molecules21010064. PMID: 26751438; PMCID: PMC6274469 (available at https://www.ncbi.nlm.nih.gov/pmc/articles/PMC6274469/). When combined, milk thistle (Silybum marianum) seed extract and prickly pear cactus (Opuntia ficus-indica) extract may exhibit amplified liver-protective effectiveness even in low dosages. The compositions of the present disclosure may use a blend of both ingredients specifically formulated to address the liver's challenge of metabolizing alcohol and reducing the ethanol-related stress on the body.

In some cases, the composition of the present disclosure may include safflower oil. The safflower oil may increase the bioavailability of milk thistle (Silybum marianum) seed extract and prickly pear cactus (Opuntia ficus-indica) extract. Thus, by using safflower oil, the composition may include effective dosages of milk thistle (Silybum marianum) seed extract and prickly pear cactus (Opuntia ficus-indica) in a small gummy format.

Prickly pear cactus extract and milk thistle extract are both known for their liver-protective and detoxifying properties, which are beneficial in combating hangover symptoms. Prickly pear cactus extract contains antioxidants and anti-inflammatory compounds that help reduce the oxidative stress and inflammation in the liver caused by alcohol consumption. This extract has been studied for its ability to reduce the severity of hangover symptoms, particularly by decreasing the inflammatory response to alcohol intake.

Milk thistle extract, which is rich in silymarin, a group of compounds said to have antioxidant and anti-inflammatory effects, is commonly used to support liver health. Silymarin is believed to protect liver cells from toxins and aid in the regeneration of damaged liver tissue. It also may help to stabilize cell membranes, preventing the uptake of toxins, and accelerate the recovery of liver cells.

When used together in a nutraceutical composition, prickly pear cactus extract and milk thistle extract can provide a synergistic effect that enhances liver function, supports detoxification processes, and reduces the impact of hangover symptoms such as headache, nausea, and lethargy. By supporting the liver's ability to process and eliminate the toxic by-products of alcohol metabolism, these extracts contribute to a more effective hangover remedy.

### Ginger Root

In some cases, the composition of the present disclosure may include ginger root (Zingiber officinale) extract. Ginger root (Zingiber officinale) extract is regarded for its anti-inflammatory and antioxidant properties, which can be beneficial for reducing inflammatory responses and oxidative stress associated with alcohol consumption. See, for instance, Wang F, Li Y, Zhang Y J, Zhou Y, Li S, Li H B. Natural Products for the Prevention and Treatment of Hangover and Alcohol Use Disorder. Molecules. 2016 Jan. 7; 21(1):64. doi: 10.3390/molecules21010064. PMID: 26751438; PMCID: PMC6274469 (available at https://www.ncbi.nlm.nih.gov/pmc/articles/PMC6274469/). Ginger root may be used to alleviate nausea and digestive issues, common symptoms associated with hangovers. The compositions of the present disclosure may include ginger root dosages between 40-60 mg to support nausea from alcohol consumption.

Ginger root extract, known for its anti-inflammatory and antioxidant properties, plays a complementary role in the nutraceutical composition by addressing several symptoms associated with hangovers. Its anti-inflammatory effects can help reduce the inflammatory response that may be triggered by alcohol consumption, while its antioxidant properties combat oxidative stress, which is another consequence of excessive alcohol intake.

Ginger is particularly renowned for its ability to alleviate gastrointestinal discomfort, such as nausea and digestive issues, which are common symptoms of a hangover. When integrated with other ingredients in the composition, such as DHM, milk thistle, and prickly pear cactus extract, ginger root extract contributes to a multifaceted approach to hangover management. DHM helps to lower blood alcohol levels and protect liver cells, milk thistle and prickly pear cactus provide liver support through their detoxifying and antioxidant effects, and the amino acid L-alanyl-L-glutamine aids in preventing disruptions to sleep patterns post-alcohol consumption.

By working in synergy with these components, ginger root extract enhances the overall effectiveness of the composition in mitigating hangover symptoms, promoting liver health, and aiding in the recovery process after alcohol consumption.

### L-Alanyl-L-Glutamine

In some cases, the compositions of the present disclosure may include L-Alanyl-L-Glutamine. The L-Alanyl-L-Glutamine may help prevent hangovers. When alcohol is consumed, the body's production of glutamine, a key amino acid, is usually inhibited. Once the consumption of alcohol stops, the body usually tries to compensate for this suppression by increasing its production of glutamine, a phenomenon known as “glutamine rebound.” See, for instance, Devenney, Lydia E., Kieran B. Coyle, Thomas Roth, and Joris C. Verster. 2019. “Sleep after Heavy Alcohol Consumption and Physical Activity Levels during Alcohol Hangover” Journal of Clinical Medicine 8, no. 5: 752 (available at https://doi.org/10.3390/jcm8050752). During this “glutamine rebound”, increased waking & light sleeping occurs during the sleep cycle, thereby preventing quality recovery during sleep. In some cases, the compositions of the present disclosure may include L-Alanyl-L-Glutamine in dosages of 40-60 mg, to prevent the intensity of this rebound and promote quality sleep and recovery.

Thus, L-Alanyl-L-Glutamine as an ingredient in the composition plays a role in mitigating the effects of “glutamine rebound,” which occurs after alcohol consumption. When alcohol is ingested, it suppresses the body's natural production of glutamine, a pivotal amino acid. Upon cessation of drinking, the body attempts to compensate for this suppression by ramping up glutamine production, leading to an increase in wakefulness and a reduction in the quality of sleep. This can result in a feeling of fatigue and unrestedness the next day. The inclusion of L-Alanyl-L-Glutamine in the nutraceutical composition provides a stable form of glutamine that may help to normalize the levels of this amino acid in the body, thereby reducing the intensity of the glutamine rebound effect. This can promote more restful sleep and a better sense of well-being upon waking, contributing to the overall effectiveness of the composition in alleviating hangover symptoms.

### Vitamin B Complex

In some cases, the compositions of the present disclosure may include vitamin B6 (pyridoxine hydrochloride) and B12 (methylcobalamin). In some cases, vitamin B6 and B12 in the composition may replenish nutrients and prevent next-day fatigue. At ratios of 4.64-6.96 mg (B6) and 232-348 mcg (B12), this vitamin B complex may support metabolic processes and overall energy levels.

The Vitamin B complex within the nutraceutical composition, which includes vitamins such as B6 and B12, works synergistically with other ingredients to enhance the body's recovery from alcohol consumption. These vitamins are integral in replenishing nutrients that are often depleted due to the diuretic effect of alcohol, which can lead to a loss of water-soluble vitamins. Vitamin B6 (pyridoxine) is involved in the metabolism of amino acids and neurotransmitters, which can help in reducing symptoms of a hangover, while Vitamin B12 (cyanocobalamin or methylcobalamin) is pivotal for maintaining healthy nerve cells and aiding in the production of DNA and RNA.

When combined with liver detoxification compounds like DHM, milk thistle, and prickly pear cactus extract, the Vitamin B complex supports the liver's metabolic processes, aiding in the faster breakdown and elimination of alcohol and its toxic by-products. This synergistic action helps to reduce the overall stress on the liver and supports its detoxifying role. Additionally, the inclusion of electrolytes in the composition works with the Vitamin B complex to maintain hydration and electrolyte balance, which is often disrupted by alcohol consumption. Together, these components form a comprehensive approach to mitigating the physiological stress of alcohol consumption and supporting overall liver function and energy levels.

### Coating

In some cases, the compositions of the present disclosure may include a combination of safflower oil and carnauba wax. In some cases, the safflower oil and carnauba wax may be a coating. The coating may be useful to preserve active ingredients of a gummy nutraceutical. Safflower oil, known for its oxidative stability, may ensures that the gummy remains effective over time as the oil forms a barrier to prevent air and moisture exposure. Safflower oil has historically been utilized in traditional remedies to increase the effectiveness of herbal compounds, potentially increasing the efficacy of our active ingredients. Carnauba wax has historical been used in preserving the integrity of perishable goods, and its strategic usage in the coating may protect sensitive ingredients from environmental degradation. Moreover, carnauba wax may offer a smooth, glossy finish that allows the gummy to not stick to teeth or packaging. Thus, in some cases, the two elements of the coating may create a synergistic effect together, with the oxidative stability of the oil being complemented by the texture-improving qualities of the wax, culminating in a coating that enhances the stability, absorption, and appeal of the gummy.

In some cases, the composition (e.g., gummy) of the present disclosure may encompass a unique formulation comprising a synergistic blend of active ingredients, essential vitamins, and a specialized coating. This formulation has been demonstrably effective, based on anecdotal evidence, in mitigating and preventing symptoms commonly associated with the consumption of alcohol, specifically those symptoms colloquially referred to as a ‘hangover.’ The formulation has been highly effective in the context of hangover prevention and symptom alleviation.

The nutraceutical composition synergistically combines electrolytes, vitamin B complexes, and liver detoxification compounds to counteract the effects of alcohol consumption. Electrolytes such as calcium, magnesium, potassium, phosphorous, sodium, and chloride are integral for maintaining hydration and electrolyte balance disrupted by alcohol intake. The vitamin B complexes, including B6 and B12, are pivotal in replenishing nutrients depleted by alcohol metabolism and in mitigating fatigue. The liver detoxification compounds, which include Ampelopsis grossedentata extract (DHM), prickly pear cactus extract, milk thistle extract, ginger root extract, and L-alanyl-L-glutamine, work in concert to enhance the liver's ability to process and eliminate acetaldehyde, the toxic metabolite of alcohol. DHM aids in reducing blood alcohol levels and protecting liver cells, while prickly pear cactus and milk thistle provide antioxidant and anti-inflammatory support for liver health. Ginger root extract contributes its anti-inflammatory and anti-nausea properties, and L-alanyl-L-glutamine helps to regulate the body's glutamine levels, promoting better sleep and recovery. Together, these components form a comprehensive approach to mitigating the physiological stress of alcohol consumption and supporting overall liver function.

### Effective Amount

Generally, where an ingredient is listed with an amount or a range of a composition (e.g., gummy), the subject ingredient may be provided in various forms, such as powders or extracts, and the like (as the case may be). Moreover, a “ingredient” may be an “effective amount” (or alternately, a “therapeutically effective amount” (or ranges)) for a targeted dose (e.g., for absorption) alone or combined with other “ingredients.” In some cases, an “effective amount” is an amount that alone, or together with further doses or other ingredients, produces a desired (therapeutic) response. The (therapeutically) effective amount to be used will depend, for example, upon the therapeutic objectives and the condition of the user.

The compositions (e.g., gummies) described herein are therefore administered by oral consumption in an effective amount to produce the desired response. Examples of such responses include (increases or decreases in) thirst, fatigue, headache, dizziness/faintness, loss of appetite, stomach ache, nausea, poor and/or decreased sleep, and elevated heart rate, and the like.

In some cases, wherever an ingredient is mentioned, the ingredient may include or be replaced by a derivative thereof, or ingredients that provide similar functional benefits.

### Gummy

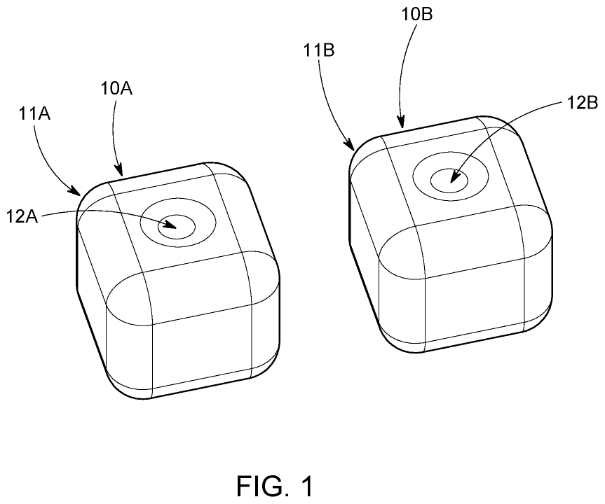

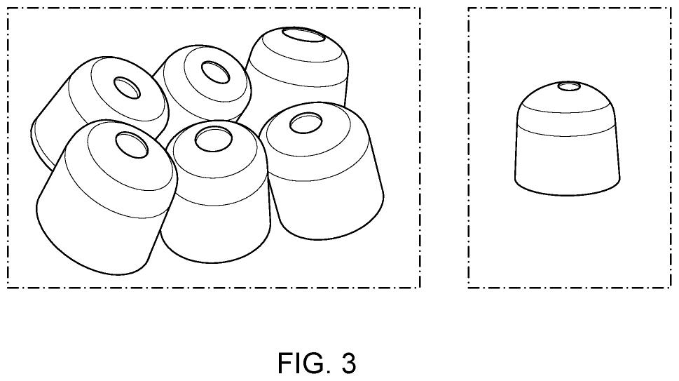

Referring now to the Figures, two representative gummy-based products 10A, 10B are shown in FIG. 1. Each gummy-based product 10A, 10B has a generally square shape, in plan view from the top, with rounded corners 11A, 11B and a centrally located circular upper surface depression 12A, 12B. Also, each gummy-based product 10A, 10B is otherwise sized and configured to resemble popular lozenges taken by people to counteract coughs and colds. Any size or shape for a gummy-based product of the present subject matter—which a person can take orally—is, of course, within the scope of the present disclosure. For example, FIG. 3 depicts an alternative version to the gummy-based product 10A, 10B.

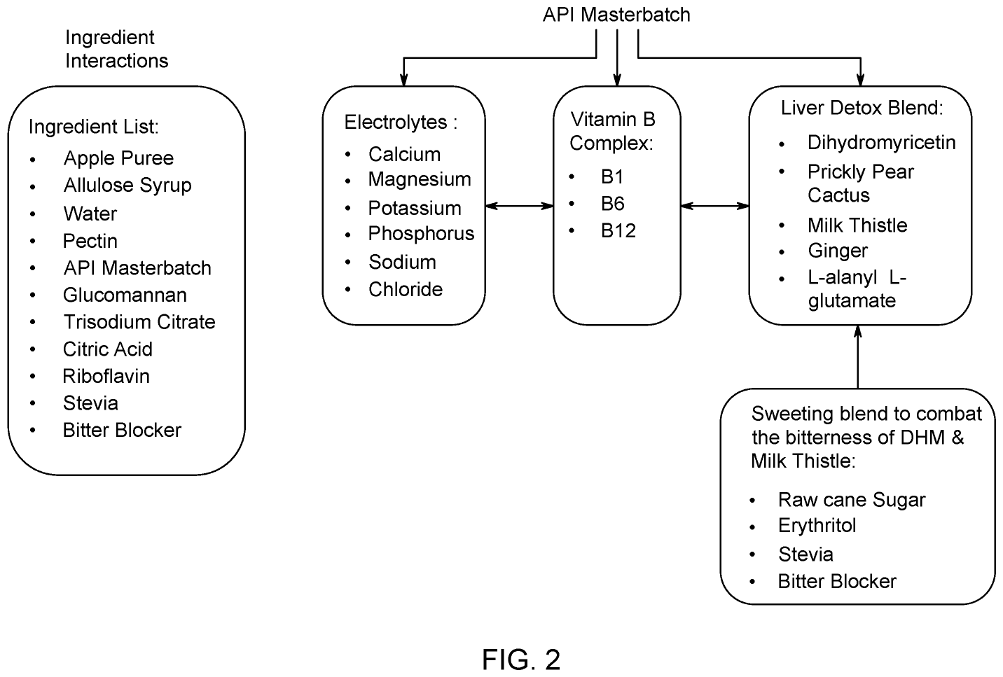

FIG. 2 illustrates ingredients of the gummy-based products 10A, 10B in an Active Pharmaceutical Ingredient (API) Masterbatch. As shown, a liver detox blend and electrolytes in the masterbatch may both interact with vitamins in the masterbatch. Sweeteners interact with the liver detox blend.

The inclusion of sweeteners in the nutraceutical composition plays a pivotal role in enhancing the palatability of the gummy by counteracting the inherent bitterness of active ingredients such as DHM (dihydromyricetin) and milk thistle extract. Sweeteners like allulose syrup, erythritol, and stevia are known for their ability to impart a pleasant sweetness without contributing excessive calories or affecting blood sugar levels. These sweeteners work by engaging the sweet taste receptors on the tongue, effectively masking the bitter taste molecules from DHM and milk thistle. Additionally, the formulation may include a bitter blocker, which further inhibits the perception of bitterness, ensuring that the gummy is enjoyable to consume. This careful balance of sweetness and flavor masking allows the gummy to deliver its beneficial effects without the off-putting bitterness that might otherwise discourage consumption.

The hangover remedy, which I have incorporated into the gummy-based products of the present disclosure are not known, at the relative percentages found by me to be effective to counteract intoxicating effects of alcohol, in any currently available products.

It was only after much experimentation that I discovered a recipe enabling us to make gummy-based products effective for a major portion of the “of age” adult population.

TABLE I

| | | Weight | |
|---|---|---|---|
| Ingredient | | Amount, | Percentage |
| Number | Ingredient Details | Grams | Amounts |
| 1 | Apple Puree | 200.00 | 31.0318 |
| 2 | Allulose Syrup | 110.00 | 17.0675 |
| 3 | Raw Cane Sugar^{†} | 78.00 | 12.1024 |
| 4 | Water (distilled or deionized) | 67.50 | 10.4732 |
| 5 | Erythritol^{†} | 47.00 | 7.2925 |
| 6 | Pectin^{†} | 40.00 | 6.2064 |
| 7 | API Masterbatch | 38.50 | 5.9736 |
| 8 | Glucomannan | 17.00 | 2.6377 |
| 9 | Stevia | 17.00 | 2.6377 |
| 10 | Bitter Blocker | 17.00 | 2.6377 |
| 11 | Citric Acid | 10.00 | 1.5516 |
| 12 | Trisodium Citrate | 1.50 | 0.2327 |
| 13 | Riboflavin (for color) | 1.00 | 0.1552 |

| | | |
|---|---|---|
| TOTALS= | 644.50 | 100.0000 |

Notes: The symbol “+” denoting production of masterbatches refers to individual amounts×12. Allulose, a low-calorie epimer of monosaccharide sugar fructose, is used by certain commercial food and beverage manufacturers as a low-calorie sweetener. Erythritol, a four-carbon sugar alcohol with no optical activity, is used as a food additive and sugar substitute. Pectin is composed of complex polysaccharides which are present in the primary cell walls of a plant, and which are abundant in the green parts of an assortment of terrestrial plants. Glucomannan, a hemicellulose component found in the cell walls of some plant species, is a water-soluble polysaccharide that is considered a dietary fiber by dieticians. Stevia is a natural sweetener and sugar substitute derived from the leaves of the plant species Stevia rebaudiana and is native to Paraguay and Brazil. Prickly pear, milk thistle, DHM (dihydromyricetin), and ginger are presently commercially available in their “whole plant powder” forms. Mineral weights are measured as their respective chelates and salts.

Table I presents an ingredient formulation for a vegan variation of a gummy-based product of the present subject matter, which incorporates a great many active ingredients.

For our “gummies” (gummy-based products of the present subject matter), there are three principal ingredients: electrolytes, vitamin B complexes, and liver detoxification compounds. The electrolytes—which include but are not limited to calcium, magnesium, potassium, phosphorous, sodium, and chloride—help one's body to stay hydrated throughout periods (e.g., nights, weekends) of alcohol consumption. Vitamin B complexes—which include but are not limited to B1, B6, and B12—help a person's body to regain nutrients lost from alcohol consumption and prevent bodily fatigue from overconsumption.

The liver detoxification blend may include clinically proven ingredients to help a person's body break down acetaldehyde produced from alcohol consumption and detoxify the person's liver, protecting it for future, otherwise normal consumption of nutritious food. Illustrative ingredients of our gummy-based products—including but not limited to prickly pear cactus, milk thistle, dihydromyricetin, ginger, and L-alanyl L-glutamate—provide our gummy-based products with select ingredients of detoxification leaving consumers of our “gummies” feeling more refreshed (than would otherwise be possible) the next morning.

Other ingredients of our “gummy” products—including but not limited to water, raw cane sugar, allulose syrup, pectin, trisodium citrate, glucomannan, riboflavin, and citric acid—help maintain structural integrity of select (e.g., vegan) and other gummy products, while containing amounts of various active ingredients effective for detoxifying a person.

In some cases, the gummy product of the present disclosure may include at least the following ingredients in the ranges indicated in Table II.

TABLE II

| Ingredient | Range | |
|---|---|---|
| Ampelopsis grossedentata extract (DHM) | 200-450 | mg |
| Vitamin B6 (Pyridoxine Hydrochloride) | 4.64-6.96 | mg |
| Vitamin B12 (Methylcobalamin) | 232-348 | mcg |
| Calcium (Calcium Phosphate) | 8.4-12.6 | mg |
| Magnesium (as citrate) | 4.8-7.2 | mg |
| Potassium (as citrate) | 18-27 | mg |

In some cases, the composition (e.g., gummy) of the present disclosure may include the electrolytes. The electrolytes may include one or combinations of: calcium, magnesium, potassium, phosphorous, sodium, and chloride. In some cases, the electrolytes may help a user's body to stay hydrated throughout periods (e.g., nights and weekends) of alcohol consumption.

In some cases, the composition (e.g., gummy) of the present disclosure may include the Vitamin B complex. The Vitamin B complex may include one or combinations of: B6 and B12. The Vitamin B complex may help a user's body regain nutrients lost from alcohol consumption and prevent bodily fatigue from overconsumption.

In some cases, the composition (e.g., gummy) of the present disclosure may include the liver detoxification compounds. The liver detoxification compounds may include one or combinations of: prickly pear cactus, milk thistle, dihydromyricetin, ginger, and L-alanyl L-glutamate. The liver detoxification compounds may help a user's body break down acetaldehyde produced from alcohol consumption and detoxify the person's liver, protecting it for future, otherwise normal consumption of nutritious food.

In some cases, the composition (e.g., gummy) of the present disclosure may include one or combinations of: allulose syrup, erythritol, sucrose, apple juice, natural flavors, citric Acid, sodium hydrogen malate, monk fruit extract, stevia, and himalayan salt.

In some cases, the composition (e.g., gummy) of the present disclosure may include a base and a coating. The base may include one or both of citrus pectin and/or water. The coating may include a mixture of an oil and a wax. For instance, the oil may be a safflower oil and the wav may be a carnauba wax.

In some cases, the composition (e.g., gummy) of the present disclosure may include natural colorants. The natural colorants may provide the gummy with specific color.

### How To Make

The following example provides a detailed description of a current procedure to produce of batch of “gummy” product of the present subject matter. While the current procedure is exemplary, it is not intended to limit the scope of the claims herein.

Step 1. Weigh and transfer 165.0 grams (g) of a sugar/erythritol/pectin masterbatch into a clean 500-milliliter (mL) beaker. Step 2. Mix thoroughly with a spatula until homogeneous. Step 3. Weigh and transfer 110.0 g of the allulose syrup int the sugar/pectin beaker. Mix with a spatula until a paste is formed. Step 4. Weigh and transfer water into a clean 250-ml beaker. Step 5. Weigh and transfer glucomannan into the water beaker. Do not mix. Step 6. Weigh trisodium citrate into a weigh boat. Set aside. Step 7. Weigh citric acid, apple puree, supplement masterbatch, and riboflavin into a clean 500-mL beaker. Slowly mix until the contents are homogeneous. Set aside. Step 8. Begin mixing the beaker containing the sugar/erythritol/pectin/allulose syrups. Step 9. Slowly begin heating to 70 degrees Celsius (within a range of +5 degrees Celsius). Step 10. Once temperature is reached, decrease temperature to about 40 degrees Celsius (° C.) and continue mixing. Step 11. Transfer trisodium citrate into water/glucomannan beaker. Begin mixing. Step 12. Slowly begin heating to 80° C. without exceeding 95° C. Step 13. Once material reaches temperature and/or becomes increasingly translucent, transfer all material from both beakers into a clean 1000-mL beaker. Step 14. Begin mixing 1000-ml beaker contents and begin heating contents until 100° C. (+5° C.) is reached. Step 15. Once 100° C. is reached, transfer the citric acid/apple puree/supplement masterbatch/riboflavin into a beaker. Step 16. Continue mixing for 15 minutes at temperature. Step 17. Turn off heat and carefully transfer material to molding sheet. Let sit at room temperature (about 23 to about 27° C.) for 30 minutes. Step 18. Transfer molding sheet to refrigerator. Refrigerate for about 3 hours. Step 19. Remove from refrigerator. Let it sit at room temperature for a minimum of 30 minutes. Step 20. If necessary, transfer to dehydration chamber for about 24 hours.

### How to Use

Young adults and individual at the age of 40 and up would consume our gummy products, as needed, through oral consumption, to reduce symptoms experienced from overexposure to alcohol and dehydration. To achieve desired relief, gummy product consumers should orally take three gummies of present size and active ingredient concentration for every 3-5 drinks (e.g., 4 fluid ounces per drink) consumed per evening, to feel refreshed and less physically fatigued (due to alcohol consumption) in the morning.

### Current Advantages and Features Including a Proposed Product Label

While DHM is included in some of the more universally recognized products in the alcohol aid market (to provide consumers with, e.g., “morning recovery” cheerfulness), they are available in hard-to-swallow pills, powders that are difficult to use in a public setting, and liquids/tablets that are not as palatable as an ordinary consumer would like. Our product therefore overcomes difficulties associated with consuming or public usage.

A first product label for a container for gummy products is shown in Table III.

TABLE III
SUPPLEMENT FACTS

| | Servings per container: 90 | | |
|---|---|---|---|
| Serving size: 3 gummies | Amount per | | |
| Calories: 17 | serving | | % DV/RDI |
| Total Fat | 0 | g | — |
| Saturated Fat | 0 | g | — |
| Trans Fat | 0 | g | — |
| Cholesterol | 0 | mg | — |
| Total Carbohydrates | 6 | g | 2% |
| Dietary Fiber | 1 | g | 4% |
| Total Sugars | 3 | g | 6% |
| Sugar Alcohols | 3 | g | N.E. |
| Protein | 0 | g | — |
| Vitamin B6 (as pyridoxine HCL) | 10 | mg | 588% |
| Vitamin B12 (as cyanocobalamin) | 500 | mcg | 20,833% |
| Vitamin B1 (as thiamine HCL) | 50 | mg | 4,167% |
| Calcium (as carbonate, citrate) | 20 | mg | 2% |
| Magnesium (as bisglycinate, oxide) | 10 | mg | 2% |
| Potassium (as potassium phosphate) | 30 | mg | 1% |
| Phosphorous (as potassium phosphate) | 10 | mg | 1% |
| Sodium (as sodium chloride) | 8 | mg | <1% |
| Chloride (as sodium chloride) | 19 | mg | 1% |
| Prickly Pear Cactus (Opuntia ficus- | 100 | mg | N.E. |
| indica) leaf | | | |
| Milk Thistle (Silybum Marianum) | 80 | mg | N.E. |
| (minimum 80% silymarin) | | | |
| Ampelopsis grossedentata extract | 300 | mg | N.E. |
| (minimum 98% dihydromyricetin) | | | |
| Ginger (Zingiber officinale) (5% | 75 | mg | N.E. |
| minimum gingerols and shogaol) | | | |
| L-alanyl-L-glutamine | 50 | mg | N.E. |

Notes:
The Percentage (%) Daily Value (DV) and Reference Daily Intake (RDI) values achieved by taking the gummy product serving size noted are listed.
N.E. indicates RDI is not established.

A second product label for a container for gummy products is shown in Table IV.

TABLE IV

Supplement Facts
Serving Size: 3 gummies
Servings Per Container: 2 servings
per pouch
Calories: 30 per serving
Amount Per Serving - % Daily
Value
Total Carbohydrate: 8 g
Total Sugars: 5 g
Includes 4 g Added Sugars
Dietary Fiber: 0.22 g
Vitamin B6 (Pyridoxine
Hydrochloride): 5.80 mg
Vitamin B12 (Methylcobalamin):
290.00 mcg
Calcium (as Calcium Phosphate):
10.50 mg †
Phosphorus (as Calcium
Phosphate): 5.40 mg †
Magnesium (as Magnesium Citrate):
6.00 mg †
Potassium (as Potassium Citrate):
22.50 mg †
Sodium (as Sodium Citrate): 2.00
mg †
Chloride (as Sodium Chloride): 3.05
mg †
LiQure Blend: 400 mg †
Prickly Pear (Opuntia ficus-Indica) Stem Extract
Milk Thistle (Silybum marianum) Seed Extract
Vine Tea (Ampelopsis Grossedentata) Leaf &
Stem Extract (std. Dihydromyricetin)
Ginger (Zingiber officinale) Root Extract
L-Alanyl-L-Glutamine
Other Ingredients: Liquid Glucose, Sucrose, Water, Citrus Pectin, Apple Juice, Natural Flavors, Natural Colors, Citric Acid, Sodium Hydrogen Malate, Monk Fruit Extract, Stevia Extract, Himalayan Salt, Coating (Safflower Oil, Carnauba Wax)

What has been illustrated and described in detail in this patent application is a gummy-based product to be taken orally by a person to counteract intoxicating effects of alcohol-containing beverages. While the present subject matter has been described with reference to exemplary embodiments, the present disclosure is not limited to these examples. On the contrary, many alternatives, changes, and modifications will become apparent to a person of ordinary skill in the art (“POSITA”) after this application is reviewed. Thus, all alternatives, changes, or modifications are to be treated as being part of the present disclosure insofar as they fall within the spirit and scope of appended claims.

### Terminology

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of +10% in a stated value.

The term “exemplary” is used in the sense of “example” rather than “ideal.” As used herein, the singular forms “a,” “an,” and “the” include plural reference unless the context dictates otherwise.

### Examples

Exemplary embodiments of the systems and methods disclosed herein are described in the numbered paragraphs below.

A1. A nutraceutical gummy composition for counteracting effects of alcohol-containing beverages and solid food products, comprising:
electrolytes;
vitamin B complexes;
liver detoxification compounds;
wherein the composition includes ampelopsis grossedentata extract (DHM), prickly pear cactus extract, milk thistle extract, ginger root extract, and L-alanyl-L-glutamine.

A2. The nutraceutical gummy composition of A1, wherein the electrolytes include one or combinations of: calcium, magnesium, potassium, phosphorous, sodium, and chloride.

A3. The nutraceutical gummy composition of A2, wherein the electrolytes help a user's body to stay hydrated throughout periods of alcohol consumption.

A4. The nutraceutical gummy composition of any of A1-A3, wherein the Vitamin B complexes include one or combinations of: B6 and B12.

A5. The nutraceutical gummy composition of A4, wherein the Vitamin B complexes help a user's body to regain nutrients lost from alcohol consumption and prevent bodily fatigue from overconsumption.

A6. The nutraceutical gummy composition of any of A1-A5, wherein the composition includes a gummy base and a coating, wherein the gummy base includes citrus pectin and water, and the coating includes safflower oil and carnauba wax.

A7. The nutraceutical composition of any of A1-A6, wherein the liver detoxification compounds help a user's body break down acetaldehyde produced from alcohol consumption and detoxify the person's liver, protecting it for future, otherwise normal consumption of nutritious food.

A8. The nutraceutical composition of any of A1-A7, wherein the composition further includes one or combinations of: allulose syrup, erythritol, sucrose, apple juice, natural flavors, citric acid, sodium hydrogen malate, monk fruit extract, stevia, and Himalayan salt.

A9. The nutraceutical composition of any of A1-A8, wherein the composition is colored with natural colorants.

A10. The nutraceutical composition of any of A1-A9, wherein the composition includes one or more of the following ranges: Ampelopsis grossedentata extract (DHM) 200-450 mg, Vitamin B6 (Pyridoxine Hydrochloride) 4.64-6.96 mg, Vitamin B12 (Methylcobalamin) 232-348 mcg, Calcium (Calcium Phosphate) 8.4-12.6 mg, Magnesium (as citrate) 4.8-7.2 mg, Potassium (as citrate) 18-27 mg, Phosphorous (Calcium Phosphate) 4.32-6.48 mg, Sodium (as sodium citrate) 1.6-2.4 mg, Chloride (as sodium chloride) 2.44-3.66 mg, Prickly Pear Cactus (Opuntia ficus-indica) Extract 50-90 mg, Milk Thistle (Silybum marianum) Seed Extract 50-90 mg, Ginger Root (Zingiber officinale) Extract 40-60 mg, L-alanyl-L-glutamine 40-60 mg.

A11. The nutraceutical composition of any of A1-A10, wherein the L-alanyl-L-glutamine reduces a glutamine rebound of the user.

A12. A nutraceutical composition for counteracting effects of alcohol-containing beverages and solid food products, comprising:
electrolytes;
vitamin B complexes;
liver detoxification compounds;
wherein the composition includes ampelopsis grossedentata extract (DHM), prickly pear cactus extract, milk thistle extract, ginger root extract, and L-alanyl-L-glutamine, and
the composition includes a gummy base and a coating, wherein the gummy base includes citrus pectin and water, and the coating includes an oil and a wax.

A13. The nutraceutical composition of A12, wherein the electrolytes include one or combinations of: calcium, magnesium, potassium, phosphorous, sodium, and chloride.

A14. The nutraceutical composition of any of A12-A13, wherein the Vitamin B complexes include one or combinations of: B6 and B12.

A15. The nutraceutical composition of any of A12-A14, wherein the oil is a safflower oil, and the wax is a carnauba wax.

A16. The nutraceutical composition of any of A12-A15, wherein the L-alanyl-L-glutamine reduces a glutamine rebound of a user.

A17. The nutraceutical composition of any of A12-A16, wherein the composition includes one or more of the following ranges: Ampelopsis grossedentata extract (DHM) 200-450 mg, Vitamin B6 (Pyridoxine Hydrochloride) 4.64-6.96 mg, Vitamin B12 (Methylcobalamin) 232-348 mcg, Calcium (Calcium Phosphate) 8.4-12.6 mg, Magnesium (as citrate) 4.8-7.2 mg, Potassium (as citrate) 18-27 mg.

A18. A method of treating effects of alcohol-containing beverages and solid food products in a subject, the method comprising causing a subject to consume a nutraceutical gummy composition, wherein the nutraceutical gummy composition includes:
electrolytes;
vitamin B complexes;
liver detoxification compounds;
wherein the composition includes ampelopsis grossedentata extract, prickly pear cactus extract, milk thistle extract, ginger root extract, and L-alanyl-L-glutamine, and
an amount of the ampelopsis grossedentata extract (minimum 98% dihydromyricetin) is an effective amount for counteracting intoxicating effects.

A19. The method of A18, wherein the composition includes a gummy base and a coating, wherein the gummy base includes citrus pectin and water, and the coating includes an oil and a wax.

A20. The method of A19, wherein the L-alanyl-L-glutamine reduces a glutamine rebound of a user.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A nutraceutical gummy composition for counteracting effects of alcohol-containing beverages and solid food products, comprising:
electrolytes;
vitamin B complexes;
liver detoxification compounds;
wherein the composition includes ampelopsis grossedentata extract (DHM), prickly pear cactus extract, milk thistle extract, ginger root extract, and L-alanyl-L-glutamine.

2. The nutraceutical gummy composition of claim 1, wherein the electrolytes include one or combinations of: calcium, magnesium, potassium, phosphorous, sodium, and chloride.

3. The nutraceutical gummy composition of claim 2, wherein the electrolytes help a user's body to stay hydrated throughout periods of alcohol consumption.

4. The nutraceutical gummy composition of claim 1, wherein the Vitamin B complexes include one or combinations of: B6 and B12.

5. The nutraceutical gummy composition of claim 4, wherein the Vitamin B complexes help a user's body to regain nutrients lost from alcohol consumption and prevent bodily fatigue from overconsumption.

6. The nutraceutical gummy composition of claim 1, wherein the composition includes a gummy base and a coating, wherein the gummy base includes citrus pectin and water, and the coating includes safflower oil and carnauba wax.

7. The nutraceutical composition of claim 1, wherein the liver detoxification compounds help a user's body break down acetaldehyde produced from alcohol consumption and detoxify the person's liver, protecting it for future, otherwise normal consumption of nutritious food.

8. The nutraceutical composition of claim 1, wherein the composition further includes one or combinations of: allulose syrup, erythritol, sucrose, apple juice, natural flavors, citric acid, sodium hydrogen malate, monk fruit extract, stevia, and Himalayan salt.

9. The nutraceutical composition of claim 1, wherein the composition is colored with natural colorants.

10. The nutraceutical composition of claim 1, wherein the composition includes one or more of the following ranges: Ampelopsis grossedentata extract (DHM) 200-450 mg, Vitamin B6 (Pyridoxine Hydrochloride) 4.64-6.96 mg, Vitamin B12 (Methylcobalamin) 232-348 mcg, Calcium (Calcium Phosphate) 8.4-12.6 mg, Magnesium (as citrate) 4.8-7.2 mg, Potassium (as citrate) 18-27 mg, Phosphorous (Calcium Phosphate) 4.32-6.48 mg, Sodium (as sodium citrate) 1.6-2.4 mg, Chloride (as sodium chloride) 2.44-3.66 mg, Prickly Pear Cactus (Opuntia ficus-indica) Extract 50-90 mg, Milk Thistle (Silybum marianum) Seed Extract 50-90 mg, Ginger Root (Zingiber officinale) Extract 40-60 mg, L-alanyl-L-glutamine 40-60 mg.

11. The nutraceutical composition of claim 1, wherein the L-alanyl-L-glutamine reduces a glutamine rebound of the user.

12. A nutraceutical composition for counteracting effects of alcohol-containing beverages and solid food products, comprising:
electrolytes;
vitamin B complexes;
liver detoxification compounds;
wherein the composition includes ampelopsis grossedentata extract (DHM), prickly pear cactus extract, milk thistle extract, ginger root extract, and L-alanyl-L-glutamine, and
the composition includes a gummy base and a coating, wherein the gummy base includes citrus pectin and water, and the coating includes an oil and a wax.

13. The nutraceutical composition of claim 12, wherein the electrolytes include one or combinations of: calcium, magnesium, potassium, phosphorous, sodium, and chloride.

14. The nutraceutical composition of claim 12, wherein the Vitamin B complexes include one or combinations of: B6 and B12.

15. The nutraceutical composition of claim 12, wherein the oil is a safflower oil, and the wax is a carnauba wax.

16. The nutraceutical composition of claim 12, wherein the L-alanyl-L-glutamine reduces a glutamine rebound of a user.

17. The nutraceutical composition of claim 12, wherein the composition includes one or more of the following ranges: Ampelopsis grossedentata extract (DHM) 200-450 mg, Vitamin B6 (Pyridoxine Hydrochloride) 4.64-6.96 mg, Vitamin B12 (Methylcobalamin) 232-348 mcg, Calcium (Calcium Phosphate) 8.4-12.6 mg, Magnesium (as citrate) 4.8-7.2 mg, Potassium (as citrate) 18-27 mg.

18. A method of treating effects of alcohol-containing beverages and solid food products in a subject, the method comprising causing a subject to consume a nutraceutical gummy composition, wherein the nutraceutical gummy composition includes:
electrolytes;
vitamin B complexes;
liver detoxification compounds;
wherein the composition includes ampelopsis grossedentata extract, prickly pear cactus extract, milk thistle extract, ginger root extract, and L-alanyl-L-glutamine, and
an amount of the ampelopsis grossedentata extract (minimum 98% dihydromyricetin) is an effective amount for counteracting intoxicating effects.

19. The method of claim 18, wherein the composition includes a gummy base and a coating, wherein the gummy base includes citrus pectin and water, and the coating includes an oil and a wax.

20. The method of claim 19, wherein the L-alanyl-L-glutamine reduces a glutamine rebound of a user.
